(19) United States
(12) Patent Application Publication
(10) Pub. No.: US 2022/0313142 A1
(43) Pub. Date: Oct. 6, 2022

(54) SYSTEMS AND METHODS FOR MEASURING NEUROLOGIC FUNCTION VIA SENSORY STIMULATION

(71) Applicant: OLFAXIS, LLC, Durham, NC (US)

(72) Inventors: William D. WOOLF, Durham, NC (US); David William SMITH, Durham, NC (US); Andreas KEIL, Micanopy, FL (US)

(21) Appl. No.: 17/634,136

(22) PCT Filed: Aug. 13, 2020
(86) PCT No.: PCT/US2020/046115

§ 371 Date: Feb. 9, 2022

Related U.S. Application Data
(60) Provisional application No. 62/888,048, filed on Aug. 16, 2019.

(51) Int. Cl. A61B 5/00; A61B 5/291; A61B 5/377; G16H 20/70; G16H 50/20; A61B 5/374

(52) U.S. Cl. CPC A61B 5/4064; A61B 5/291; A61B 5/377; A61B 5/4094; A61B 5/6803; A61B 5/7267; G16H 20/70; G16H 50/20; A61B 5/374; A61B 2560/0431

(57) ABSTRACT

Systems and methods for evaluating neurologic function of a subject are described. An odorant, auditory and/or somatosensory generator is configured to deliver a sensory stimulation to the subject, a plurality of electrodes are configured to be attached to the subject, and a handheld EEG control unit is configured to control the odorant, auditory and/or somatosensory generator, process the neural signals from the plurality of electrodes and generate an assessment of neurologic function of the subject.

### RELATED APPLICATION

This application claims the benefit of and priority to U.S. Provisional Patent Application No. 62/888,048 filed Aug. 16, 2019, the disclosure of which is incorporated herein by reference as if set forth in its entirety.

### FIELD OF THE INVENTION

The present invention relates generally to systems and methods of evaluating neurologic dysfunction.

### BACKGROUND OF THE INVENTION

There is significant evidence that the sense of smell is disrupted by brain dysfunction; changes in smell are some of the best predictors of mild traumatic brain injury (mTBI) and neurodegenerative diseases (e.g., Alzheimer's and Parkinson's Diseases). Changes in smell are sensitive indicators of mTBI, even in the absence of radiographic evidence of injury.

Most of the extant scientific literature supporting the link between olfactory deficits and mTBI/neurodegenerative diseases is derived from behavioral/perceptual olfactometry studies—at present the gold standard. In some patients, however, behavioral smell tests are not possible (i.e., the patient is unconscious, uncooperative or an infant). Additionally, behavioral and cognitive tests of smell are adversely susceptible to experience, motivation, educational level, language, etc. In these subjects, electrophysiological measures may be the best alternative, measures comparable to optoacoustic emissions and/or auditory brainstem responses (ABR) tests of hearing.

Neurological measures of olfactory function (olfactory evoked potentials (OEPs) and olfactory event-related potentials (OERPs)), which can be measured using standard electrophysiological techniques, such as quantitative electroencephalographic (qEEG), are highly correlated with the behavioral measures. OEPs and OERPs can be measured using scalp EEG electrodes. Using standard EEG methods, it is also possible to simultaneously visualize cortical oscillatory responding, including alpha, beta, gamma, delta and theta frequency oscillations. It is also possible to measure changes in alpha band oscillation responding along with the OEPs and OERPs. Alpha band oscillations are generated by thalamic pacemaker cells and are present when the brain is unstimulated (i.e., is “idling”) and are believed to aid in detecting new, incoming sensory stimulation; alpha oscillations rapidly decrease (i.e., are “desynchronized”) when the brain is activated by external sensory stimuli or by cognitive testing. Changes in alpha band “desynchronization” to new stimuli is predictive of repeated concussions and mTBI.

There remains a need for systems and methods that provide objective measures of the conduction of neural information from sensory receptors in the nose through diffuse projections within the brain.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Given that functional changes in the brain are defining characteristics of mTBI, Applicant has created a novel composite index for detecting mTBI using a combination of EEG measures that directly characterize both sensory-evoked and resting cortical neural activity. A significant benefit of this technology when compared with current, single metric assessment tools, is that it provides a composite index composed of multiple, direct neurophysiological measures, each individually capable of assessing mTBI and overall brain functional status, offering the possibility of immediate, concurrent comparisons across multiple, concurrent tests to verify neurological status.

Embodiments of the present invention include systems and methods that use olfactory stimulation, through natural sensory receptors and neural pathways, to generate OEPs and OERPs (and to suppress cortical oscillations, such as alpha, beta, gamma, delta and theta waves) in conjunction with multimodal assessment using somatosensory and/or auditory stimulation. Changes in olfactory function are sensitive indicators of neurological function in and of themselves; however, by combining olfactory, somatosensory, auditory measures, with resting EEG measures of epileptiform and non-epileptiform abnormalities, this approach provides a novel and powerful electrophysiological measure of brain neural function for use in detecting mTBI and/or neurodegenerative diseases, like Parkinson's and or Alzheimer's disease, that does not require behavioral responding from the test subject.

In some embodiments, a system for measuring neurologic function of a subject, includes an odorant generator configured to deliver an odorant stimulation to the subject, and a plurality of electrodes configured to be attached to the subject at respective different locations, such as indicated by the 10/20 international EEG electrode location standard. The plurality of electrodes are configured to collect neural signals from the subject's brain as a result of the sensory stimulation (e.g., olfactory, auditory, somatosensory stimulation, etc.) and while unstimulated (i.e., “resting EEG”). The system also includes a handheld recording and control unit configured to control the odorant generator and deliver odorant stimuli and to record, analyze and process the neural signals from the plurality of electrodes and generate an assessment of neurologic function of the subject. In other embodiments, the handheld control unit is configured to transmit or otherwise electronically send the collected neural signals to a remote device for processing and evaluation by a neurological expert.

In some embodiments, the odorant generator includes or is attached to a handheld intranasal delivery assembly for delivering a monorhinal or birhinal odorant in order to activate olfactory receptors and olfactory cortical neurons. In other embodiments, the intranasal delivery assembly includes a mask configured to be placed over the nose or the face of the subject.

By stimulating simultaneously, individually or sequentially, other sensory systems such and auditory and somatosensory in addition to the olfactory system, the normal or dysfunction of other brain systems and cortical regions may be assessed.

In some embodiments, the system also includes an auditory sound generator configured to deliver an audible stimulation to the subject, for example via one or more earbuds worn by the subject. However, other types of audio devices may be utilized.

In some embodiments, the system may also include a vibrotactile stimulator configured to generate a somatosensory stimulation to the subject. For example, the vibrotactile stimulator may be configured to generate a somatosensory stimulation to skin of the subject. In some embodiments, somatosensory stimulation may be generated via electrical stimulation, such as electrodes attached to the skin of the subject.

According to some embodiments of the present invention, a method of measuring neurologic function of a subject includes delivering an odorant stimulation to the subject via an odorant generator, collecting neural signals from the subject via a plurality of electrodes attached to the subject at respective different locations, wherein the neural signals are generated by the odorant stimulation, and processing the neural signals from the plurality of electrodes and generating an assessment of neurologic function of the subject. The neural signals may include neurological measurements of olfactory evoked potentials (OEPs) and olfactory event-related potentials (OERPs). In addition, the neural signals may include neurological measurements of epileptiform and non-epileptiform abnormal cortical activity. A blow to the head can also cause direct, mechanical damage to cortical neurons, as well as secondary neuronal insult by disruption of normal ionic and/or metabolic function. This damage can affect brain inhibitory/excitatory mechanisms and can be detected in the presence of epileptiform and non-epileptiform abnormal neural activity.

In some embodiments, delivering the odorant stimulation and processing the neural signals from the plurality of electrodes is performed by a handheld control unit.

In some embodiments, delivering the odorant stimulation comprises delivering the odorant stimulation monorhinally or birhinally. For example, an odorant generator used to deliver the odorant stimulation may include a handheld intranasal delivery assembly for delivering a monorhinal or birhinal odorant. In other embodiments, the intranasal delivery assembly may include a mask configured to be placed over the nose or the face of the subject.

In some embodiments, an audible stimulation may be delivered to the subject with the odorant stimulation, and the neural signals collected from the subject are generated by the odorant stimulation and the audible stimulation.

In some embodiments, a somatosensory stimulation may be delivered to the subject with the odorant stimulation, and the neural signals collected from the subject are generated by the odorant stimulation and the somatosensory stimulation.

In some embodiments, a somatosensory stimulation and an audible stimulation may be delivered to the subject with the odorant stimulation, and the neural signals collected from the subject are generated by the odorant stimulation and the somatosensory and the audible stimulation.

In some embodiments, processing the neural signals from the plurality of electrodes and generating an assessment of neurologic function of the subject comprises comparing a composite index of neural signals for the subject to a previous baseline composite index obtained from the subject. In some embodiments, processing the neural signals from the plurality of electrodes and generating an assessment of neurologic function of the subject comprises comparing a composite index of neural signals for the subject to a normative composite index of olfactory and cortical measurements of known neurologic function. The use of the composite index provides a way for determining if the subject is mTBI afflicted, unafflicted, or is in need of further evaluation.

Embodiments of the present invention are advantageous because olfactory electrophysiological and resting EEG responses can be measured in uncooperative (e.g., infants or malingers) or unconscious subjects and are unaffected by unintentional biases caused by motivation, educational level and/or language. OEPs are responses in olfactory peripheral neurons, and OERPs are delayed responses of cortical and higher level neurons to olfactory stimulation, as the neural activity is conducted from the periphery to CNS. The presence of OERPs can also be verified as changes in cortical alpha frequency band oscillation power or frequency following odorant stimulation. Changes in alpha frequency band power or frequency can be produced by sensory (including by odorant stimulation) motor or cognitive stimulation. Changes in alpha frequency band oscillations with stimulation or motor or cognitive stimulation is called “alpha desynchronization.” Alpha desynchronization responses have been shown to change following concussions and mTBI. By using OERPs, it may be possible to assess higher level, cognitive function/dysfunction.

Inclusion of auditory and somatosensory stimulation, either before, simultaneous with, or following odorant stimulation will allow assessment of function in wider brain regions. Embodiments of the present invention are advantageous because, by integrating multisensory stimulation, a more comprehensive assessment of brain function to diagnose and monitor mTBI can be obtained.

Embodiments of the present invention are advantageous because, by including resting EEG measures, epileptiform and non-epileptiform EEG abnormalities can be detected. Epileptiform and non-epileptiform neural activity is common following a concussive blow to the head.

Embodiments of the present invention include a small, handheld, fully automated, cutting-edge multi-modal capability for diagnosing mTBI and analytics to compute an assessment index: the Olfaxis Composite Index (OCI) uses a combination of a number of olfactory and epileptiform and non-epileptiform abnormal EEG metrics, each individually shown to be a sensitive indicator of mTBI, thereby avoiding a single measure failure point approach. The OCI may also use auditory and/or somatosensory metrics in some embodiments. A system according to embodiments of the present invention is intended as a replacement for current self-reporting and computer-based neurocognitive assessment tools. It uses quantitative EEG to calculate olfactory and cortical brain resting EEG measures; these measures are unaffected by patient demographic or cooperation. This allows the creation of an objective OCI measure for detecting mTBI and to assess the presence of sub-clinical brain injuries that, among other poor outcomes, might eventually lead to early onset dementia. This OCI measure will also be invaluable for monitoring rehabilitation and recovery, and for determining “Return to Duty” (RTD) in military applications, and “Return to Play” (or “Work”) (collectively referred to as “RTW”) in civilian applications.

Applicant has developed a composite index combining CNI (e.g., the Olfactory Nerve) functional measures, detection of epileptiform and non-epileptiform abnormalities, and cortical oscillatory bandwidth measures. In an embodiment, hardware includes an international standard 10-20 system electrode cap containing a full array of twenty-one EEG scalp electrodes, a miniaturized odorant generator, and a handheld EEG-recording device. The recorder is capable of onboard analytics using the component measures of sensory and resting EEG measures to compute the OCI, and/or is capable of transmitting the recording electronically for remote, expert review. A significant benefit of the present invention when compared with current, single metric assessment tools, is that it provides an OCI composed of multiple, direct neurophysiological measures, each individually capable of assessing mTBI status, offering the possibility of immediate comparisons across multiple, concurrent tests to verify neurological status.

According to some embodiments of the present invention, a method includes the following operations performed by a processor: accessing sensory evoked response data and EEG data obtained from each of a first plurality of subjects and from each of a second plurality of subjects, wherein the first plurality of subjects are known to have mTBI, and wherein the second plurality of subjects are known to be mTBI unafflicted; training a machine learning model to identify a brain state associated with mTBI using the sensory evoked response data and EEG data, such as resting EEG data, obtained from each of the first plurality of subjects; and training the machine learning model to identify a brain state associated with non-mTBI using the sensory evoked response data and EEG data, such as resting EEG data, obtained from each of the second plurality of subjects. The sensory evoked responses are obtained from each of the first and second plurality of subjects via one or more of the following: olfactory stimulation, audible stimulation, somatosensory stimulation. Training the machine learning model to identify a brain state may include training the machine learning model to identify epileptiform and non-epileptiform abnormal cortical activity from the EEG data. The method further includes using the machine learning model to identify a brain state of a subject with an unknown mTBI condition.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIGS. 1-2 illustrate systems for measuring OEPs and OERPs using standard electroencephalographic (EEG) methods, according to some embodiments of the present invention.

FIG. 3 is a flowchart of operations for OCI testing to deliver decision-support result for assessing mTBI, according to some embodiments of the present invention.

FIG. 4 illustrates an exemplary hyperplane for use in generating an index that classifies test results as from healthy individuals, or from individuals with TBI, according to some embodiments of the present invention.

FIG. 5 illustrates an example computing system that may be utilized with embodiments of the present invention.

### DETAILED DESCRIPTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “secured”, “connected”, “attached” or “coupled” to another feature or element, it can be directly secured, directly connected, attached or coupled to the other feature or element or intervening features or elements may be present. In contrast, when a feature or element is referred to as being, for example, “directly secured”, “directly connected”, “directly attached” or “directly coupled” to another feature or element, there are no intervening features or elements present. The phrase “in communication with” refers to direct and indirect communication. Although described or shown with respect to one embodiment, the features and elements so described or shown can apply to other embodiments.

The term “circuit” refers to software embodiments or embodiments combining software and hardware aspects, features and/or components, including, for example, at least one processor and software associated therewith embedded therein and/or executable by and/or one or more Application Specific Integrated Circuits (ASICs), for programmatically directing and/or performing certain described actions, operations or method steps. The circuit can reside in one location or multiple locations, it may be integrated into one component or may be distributed, e.g., it may reside entirely or partially in a portable housing, a workstation, a computer, a pervasive computing device such as a smartphone, laptop or electronic notebook, or partially or totally in a remote location away from a local computer or processor of a respective test unit or device or a pervasive computing device such as a smartphone, laptop or electronic notebook. If the latter, a local computer and/or processor can communicate over local area networks (LAN), wide area networks (WAN) and can include a private intranet and/or the public Internet (also known as the World Wide Web or “the web” or “the Internet”). Systems and devices according to embodiments of the present invention can comprise appropriate firewalls and electronic data interchange standards for HIPPA or other regulatory compliance. In the traditional model of computing, both data and software are typically substantially or fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud”. Cloud storage may include a model of networked computer data storage where data is stored on multiple virtual servers, rather than being hosted on one or more dedicated servers. Data obtained by various systems and devices according to embodiments of the present invention can use the Cloud.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

As used herein, the term “and/or” includes any and all combinations of one or more of the associated listed items and may be abbreviated as “/”.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that although the terms first and second are used herein to describe various features or elements, these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “about”, as used herein with respect to a value or number, means that the value or number can vary by +/−twenty percent (20%).

Olfactory neural pathways, originating in the nasal cavity, reach into the central nervous system where they branch diffusely within the brain. These olfactory tracts play critical roles in the brain's most important functions, including emotion, memory and executive function. As a consequence, damage to any of these areas can result in changes in cognitive, emotional and olfactory function (cf., Osborne-Crowley, 2016; Alosco et al., 2016). Research studies have repeatedly shown a relationship between olfactory dysfunction and traumatic brain injury (TBI) (DeGuise et al., 2015; Caminiti et al., 2013; Drummond et al., 2015). Likewise, it is known that changes in olfactory function are some of the first, and most accurate predictors of the eventual onset of Parkinson's and Alzheimer's Diseases (cf., Doty, 2003; Berendse et al., 2011; Doty, 2012; Rahayel et al, 2012; Velayudhan et al., 2013; Behrman et al., 2014). The contents of these documents are hereby incorporated by reference as if recited in full herein. TBI is one of the most common causes of olfactory dysfunction, though most of the afflicted are unaware of the olfactory sensory deficit.

The term “olfactory evoked potential” (OEP) refers to the electrical neural responses generated by the response (neural receptor potentials) of olfactory receptors (in, and between, the main olfactory epithelium in the nasal cavity and the olfactory bulb in the forebrain) to odorant stimulation. OEPs can be obtained using an electrode placed in the epithelium, nasal cavity, on the surface of the bridge of the nose, or on the scalp. The term “olfactory event-related potential” (OERP) refers to the electrical neural responses generated in cortical neurons by neural electrical activity conducted from “lower” regions of the olfactory central nervous system (i.e., olfactory receptors and olfactory bulb). OERPs can be obtained from surface electrodes using standard electroencephalographic (EEG) electrodes, methods and instrumentation.

There is significant evidence that the sense of smell is disrupted by head trauma, and that changes in smell are some of the best predictors of TBI. Changes in smell are sensitive indicators of TBI, even in the absence of radiographic evidence. Changes in olfactory function are sensitive indicators of neurodegenerative diseases; because of the sensitivity, some have argued that Parkinson's Disease is an olfactory disease. Changes in olfactory function can precede the cognitive and motor changes associated with neurodegenerative diseases (e.g., Alzheimer's and Parkinson's diseases) by years or decades. Most of the data in the scientific literature supporting the link between olfactory deficits and TBI and neurodegenerative diseases are from behavioral/perceptual olfactometry studies. Electrophysiological measures of olfactory function (OEPs and OERPs) are highly correlated with the behavioral measures, but are less frequently used. The current scientific literature also suggests that the degree of olfactory dysfunction following head trauma predicts/indicates the magnitude and, possibly, the location of TBI. These data are primarily from behavioral measures.

Embodiments of the invention can provide objective electrophysiological olfactory function tests that have clinical utility and may be used for patient screening, i.e., to deliver results that inform decisions about treatment of patients, potentially in conjunction with other testing. Embodiments of the invention can use evaluation of olfactory function to assess whether a patient/user may have TBI. Degradation of olfactory function can also be a biomarker for other neurological conditions and neurodegenerative diseases.

Additional embodiments of the present invention can use multisensory stimulation, where auditory sounds and somatosensory vibrotactile or electrical stimuli on the skin are presented before, simultaneous with, or after the odorant. Multisensory stimulation will activate and assess sensory function in wider brain regions than olfactory stimulation alone.

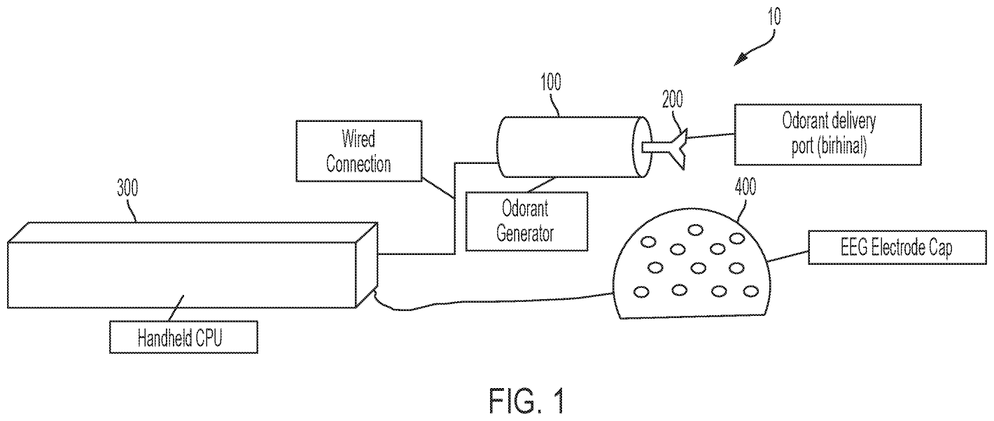

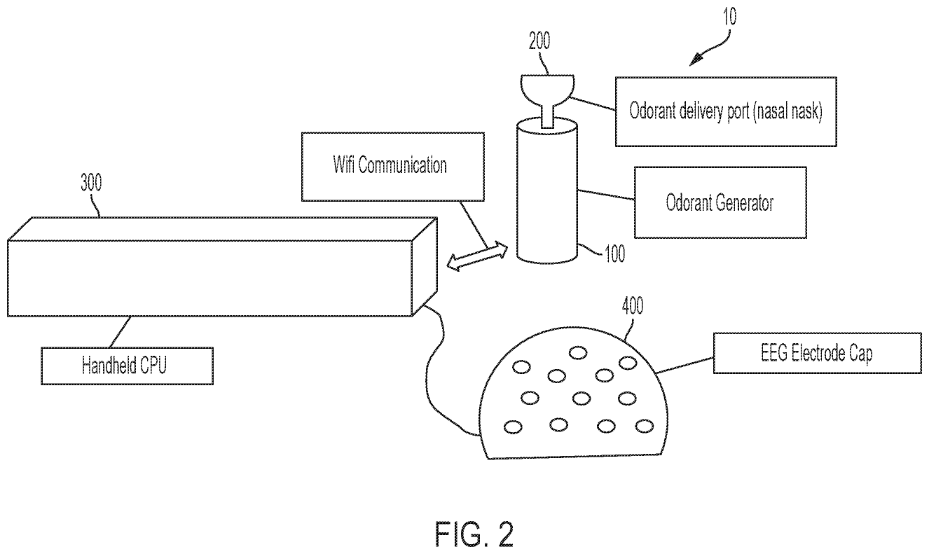

FIGS. 1-2 illustrate a system 10 for measuring OEP and OERP responses, according to some embodiments of the present invention, and that can be utilized with auditory and somatosensory stimuli generating devices. The system 10 does not require a behavioral response from the test subject, and may therefore be used on cooperative as well as nonresponsive (e.g., loss of consciousness) or uncooperative subjects (e.g., malingers or infants). The system 10 includes an odorant generator 100 for generating an odorant (e.g., phenyl ethanol, butanol, propanol, cinnamon, etc.), an intranasal delivery assembly 200 for delivering an odorant to a subject, a handheld EEG recording control unit 300, and an EEG electrode cap 400 for attachment to a subject.

The odorant generator 100 provides a puff of odorant to the intranasal delivery assembly 200, which delivers the puff of odorant to a subject. An exemplary odorant generator 100 is the Aromastic device, available from Sony Corporation, Tokyo, Japan. In some embodiments, an odorant utilized by the odorant generator 100 can comprise a gel odorant wherein the gel is held in a mesh/perforated structure, or in a polymer that can be released within a cartridge. In some embodiments, a liquid phase odorant (e.g., from ampoules, etc.) may be dispensed, prior to use/on cartridge insertion, onto an absorbent diaper-like material. In some embodiments, a cartridge that holds a single liquid odorant could be utilized. In some embodiments, a multiple reservoir cartridge that holds two or three different odorants could be utilized.

Post-traumatic epilepsy (PTE) can be a debilitating consequence of TBI, accounting for 10-20% of cases of epilepsy in the general population (Wilmore, 1990). The likelihood of developing PTE following TBI is related to the severity of the injury, and it is rare in cases of mTBI. However, epileptiform activity, a response type common in epilepsy in the absence of seizures, is observed with EEG in up to 10% of cases of mTBI (Busek and Faber, 2000; Lewine et al., 2007; Ronne-Engstrom and Winkler 2006). It has been argued that mTBI produces a disruption in normal brain excitatory-inhibitory networks, resulting in increased abnormal response patterns (Witkowski et al., 2019). Studies in humans and in animal models suggest that immediately following mTBI, the typical progression is from epileptiform activity to a generalized, overall slowing (i.e., a “non-epileptiform abnormal activity”) of the EEG traces (c.f., Dixon et al., 1987; Nilsson et al., 1994; Ronne-Engstrom and Winkler 2006; Walker, 1994). Normal EEG typically returns within six to twelve months of the mTBI.

Applicant's novel, innovative approach includes a small, handheld, fully automated, cutting-edge multi-modal capability for diagnosing mTBI. The handheld device contains onboard EEG analytics capable of computing a number of sensory and resting EEG measures, which comprise the OCI. The OCI system is a composite measure combining a number of olfactory sensory and resting EEG measures for detecting epileptiform and non-epileptiform abnormal cortical responding, thereby avoiding a single failure point approach. The system is intended as a replacement for current self-reporting and computer-based neurocognitive assessment tools. The OCI uses a quantitative EEG composite of olfactory and cortical brain resting EEG measures; these indices are unaffected by patient demographic or cooperation. This allows for the creation of an objective measure for detecting mTBI and to assess the presence of sub-clinical brain injuries that, among other poor outcomes, might eventually lead to early onset dementia (Ling et al., 2017). This measure will also be invaluable for monitoring rehabilitation and recovery, and for determining RTD and/or RTW.

The handheld EEG control unit 300 connects to and controls delivery of odorant from the odorant generator 100 via hardwire, Wi-Fi or some other electronic control method (e.g., via a Tucker Davis Lab Rat or Optima Neuroscience Cerescope). The handheld EEG control unit 300 also connects to the EEG electrode cap 400 via hardwire or Bluetooth, Wi-Fi or some other electronic control method and measures impedance of the EEG electrodes of the cap once attached to scalp of a subject. The handheld EEG control unit 300 controls testing and collects/processes/analyzes the EEG response waveform data. An exemplary EEG control unit 300 is the Cerescope instrument with existing software for standard EEG, available from Optima Neuroscience, Alachua, Fla.

The EEG electrode cap 400 uses an international standard 10-20 system electrode array and measures electroencephalographic response from the brain of the subject. Exemplary EEG caps are available from EncephaloDynamics, Inc., Gainesville, Fla. (www.eeg-now.com/online-store). The 10-20 standard electrode system dictates exact location of electrodes on the scalp, an allows any experienced neural expert to interpret the waveform data.

Sensory neural activity evoked by odorant stimuli can be measured from the electrodes of the electrode cap 400. After recording the evoked neural responses, the neural responses can be measured directly or by their effect on other brain responses, such as beta, gamma, theta and/or alpha band oscillations using quantitative electroencephalography (qEEG) analysis. The qEEG analysis can be performed using software, such as MatLAB and EEGLab. For example, when odorants are presented to the nose, they produce significant suppression or desynchronization of alpha band oscillations that can be visualized as a decrease in alpha bandwidth power or amplitude or as a “slowing” or decrease in oscillatory frequency.

EEG recording and response waveform data storage is continuous once testing of a subject begins. The entire EEG waveform is stored in the handheld EEG unit 300 for analysis. The entire EEG waveform can be transmitted for remote storage or analysis. Quantitative electroencephalography (qEEG) analysis can be automated in the handheld EEG control unit 300, or the EEG waveform can be transmitted electronically (e.g., wirelessly, via Wi-Fi, via mobile phone, etc.) to a hospital, clinic, or the like, for immediate analysis by neurological experts. Measurements that can be calculated from stored waveform using qEEG include olfactory evoked potentials (OEP), OEP amplitude as a function of odorant concentration, cortical olfactory event-related potentials, alpha band frequency, alpha band changes in frequency or power (e.g., desynchronization/resynchronization), resting EEG, for evidence of epileptiform or non-epileptiform abnormal activity, etc. Epileptiform activity, with or without seizure, is immediately evident in the EEG following TBI. The EEG waveform can also be subjected to processing to overcome potentially obscuring background physiological noise, such as eye muscle artifacts, the movement of blood, respiration etc.

The handheld EEG control unit 300 may have software for performing signal averaging and signal processing to computationally increase the OEP/OERP waveform signal-to-noise ratio and aid in response detection, calculating of OEP/OERP response latency and amplitude, and for evidence of epileptiform or non-epileptiform abnormal activity, etc. The EEG control unit 300 may also store subject medical history, responding and analytics. The handheld EEG control unit 300 may also run software to analyze EEG waveforms utilizing standard software programs such as MatLAB EEGLab (Mathworks, Natick, Mass.).

The odorant generator 100 can be controlled by the EEG control unit 300 via a wired connection (FIG. 1) or a wireless (e.g., Wi-Fi, Bluetooth, etc.) connection (FIG. 2). The odorant generator 100 includes an odorant delivery port 200, to deliver odorant directly to the nose of a subject. The odorant generator 100 can deliver odorants monorhinal (one nostril at a time, with the other nostril plugged, or not), birhinal (both nostrils simultaneously), or via a nasal mask (e.g., CPAP-like mask). Exemplary delivery devices for delivering odorants are available from Sony Corporation, Tokyo, Japan (e.g., the Aromastic personal portable aroma diffuser) and Olfaxis, LLC, Morrisville, N.C. Exemplary nasal masks are available from Philips Respironics, Amsterdam, The Netherlands (e.g., ComfortGel™ Nasal CPAP Mask). In some embodiments, the EEG electrode cap 400 can have a CPAP-like mask attached to or integrated therewith so that the mask can be placed on the subject once the EEG electrode cap 400 is secured on the subject's head.

Auditory sound stimuli created under the direction of the EEG control unit 300 is communicated to the test subject's ear by earbud transducers inserted into the external ear canal, or by an acoustic headphone speaker placed near the ear, etc. Sound stimuli may be delivered at a moderate (˜70 dB SPL) level, although various decibel levels may be utilized. Somatosensory stimuli can be created by a vibrotactile stimulator (e.g., a vibration device attached to the body, such as the back of the hand, etc.) and under control by the EEG control unit 300. Somatosensory evoked potentials can be evoked by vibrotactile stimulator, or a 0.2-2 millisecond duration electrical stimulus, delivered to surface electrodes on the medial nerve at the wrist. Tactors, such those as available from Engineering Acoustics, Inc., Casselberry, Fla., can be attached to the fingertips, and can stimulate at 60 Hz, or a single square pulse.

Concussions and mTBI can interfere with alpha band desynchronization produced by working memory tests. Working memory tests are typified by asking a person to repeat a sequence of numbers, then asking them to recall the number x or y before the last number. Working memory tests are behavioral and require active participation from the subject. These are affected by attention, education, language, cooperation, etc., or variations in test conditions or examiner expertise. Embodiments of the present invention generate evoked-sensory responses and do not require cooperation from the subject.

Concussive blows to the head are known to produce epileptiform and non-epileptiform activity in the cortex, which can be detected simultaneously with OEP and OERP responses and using the same scalp EEG electrodes and recording system. Some of the most common interictal epileptiform activity detected following mTBI includes neural spikes, sharp waves, and spike- and slow-wave complexes.

Non-epileptiform abnormal EEG activity is also common following mTBI, including intermittent slow wave (i.e., decreases in cortical wave frequency) transients and abnormal EEG synchronization. Slow wave transient responding occurs during waking or sleep states. Abnormal slow waves are persistent during waking state (also delta or theta ranged), and the term is used interchangeably with “slow waking background.”

Abnormal non-epileptiform changes in EEG synchronization are also common in mTBI. Response coherence is a measure of the amount of shared activity between two cortical regions, and changes in response phase is a measure of the timing of shared rhythms between two brain regions. Changes in coherence/phase measures reflect activity in conduction pathways between cortical regions and have been shown to be predictive of mTBI.

Given that functional changes in the brain are defining characteristics of mTBI, Applicant has created a novel composite index for detecting mTBI using a combination of EEG measures that directly characterize both sensory-evoked stimulation (e.g., olfactory, auditory and/or somatosensory stimulation) and resting cortical neural activity (epileptiform and non-epileptiform abnormalities).

Applicant's unique approach has been to create the OCI composed of a series of scientifically proven mTBI biomarkers, all mathematically derived from a single, long-duration EEG data stream. The OCI does not require additional test time to collect the numerous component biomarker measures. By using Support Vector Machine Learning, those component measures will allow the classification of test subject results as indicating a number of mTBI status states, for example, afflicted, unafflicted, or as in need of further evaluation. However, classification scales with multiple other data points may be possible. Embodiments of the present invention are not limited to “afflicted”, “unafflicted”, or “in need of further evaluation”.

The olfactory neural system originates with receptors in the olfactory epithelium in the nasal cavity. The bipolar olfactory sensory neurons then pass through the cribriform plate as the 1^{st} cranial nerve (i.e., the olfactory nerve) and terminate in the olfactory bulb in the central nervous system. It has been known for nearly 150 years, and documented in dozens of research and case studies, that CNI, as it passes through the cribriform plate, can be stretched, or severed, by a concussive blow to the head, which can then be detected as a change in olfactory sensitivity and or function.

A system according to embodiments of the present invention is the only mTBI detection system that employs natural stimulation of a sensory system, then tracks the functional neural conduction from the peripheral, into the central nervous system. A decrease in response amplitude may indicate a loss of olfactory sensitivity and the presence of mTBI.

Current neurocognitive tests for mTBI, lack the direct objectivity necessary to measure brain function as such measures are susceptible to influence by motivation, language, education, and socioeconomic variables. Using a handheld EEG, the OCI provides the objective capability to assess neurologic function near the point of injury (POI) as a direct and objective biomarker of mTBI.

Applicant's system is a novel combination of proven neuroscience and cutting-edge technologies that present an opportunity to deliver a significant and vital step forward in the assessment of mTBI. The system replaces multiple tests in the current Clinical Practice Guidelines (CPGs) for TBI that are subjective, prone to motivational or external confounds, time-consuming, and/or difficult to conduct near the point of injury (POI). The system employs a handheld, portable EEG-based recording system and utilizes a novel, composite index that combines highly sensitive and specific measures of CNI and cortical brain function to assist in the detection of mTBI, near the POI. Applicant's system is capable of monitoring treatment efficacy and recovery trajectory, as well as aiding in RTD and RTW decision making. Additionally, the system will provide data to aid in the understanding and treatment of successive concussion/mTBI events, and will document the cumulative effect of subclinical blows to the head and concussive blasts.

A strength of Applicant's composite index approach is that the handheld system collects a continuous stream of raw-EEG signal from all twenty-one sensors, covering the entire brain. All of the component biomarkers can be extracted mathematically from the same EEG data stream. This approach delivers the efficacy of assessing multiple mTBI biomarkers simultaneously.

Concussions and mTBI can be identified as either baseline-post concussion comparisons of evoked responses (e.g., as decreases in amplitude of waveforms), decreases in spread or amplitude of scalp surface response voltage gradients, changes in the speed or location of current flow from one brain location to another (i.e., current might not flow between two normal brain cortices if there is mTBI brain damage in between) as recorded by surface EEG electrodes. The same or similar changes in neural responding caused by neural or brain deterioration due to neurologic disease may be measurable, albeit over a longer lifespan period (slow progression of a neurodegenerative disease), hence a measure of “neurologic dysfunction.” Concussions and mTBI (and NDs) might also be identified by comparing a single measure to a normative population database.

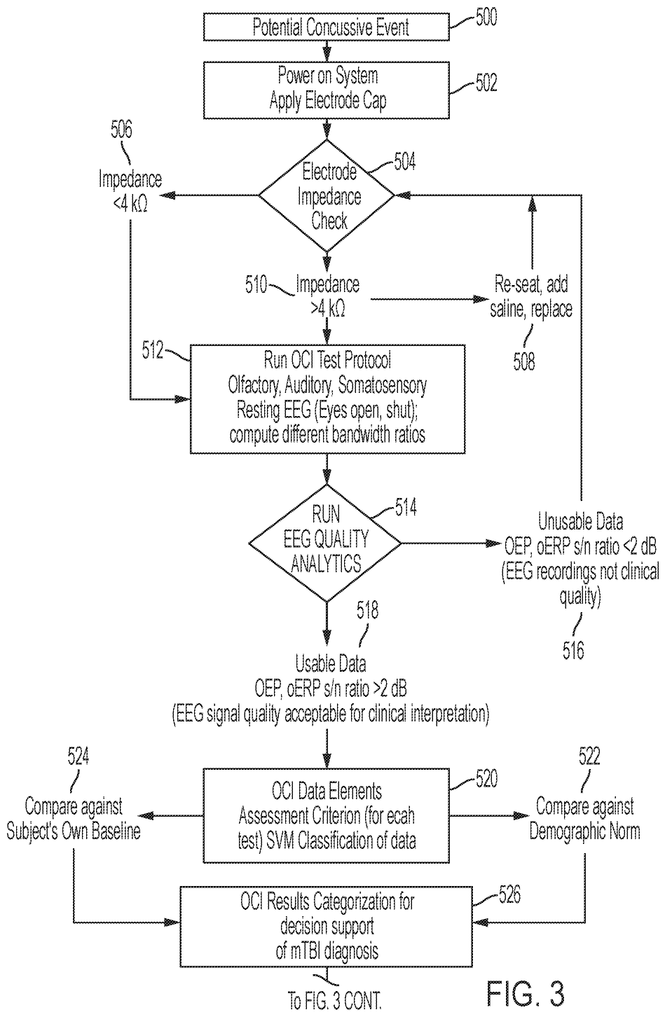

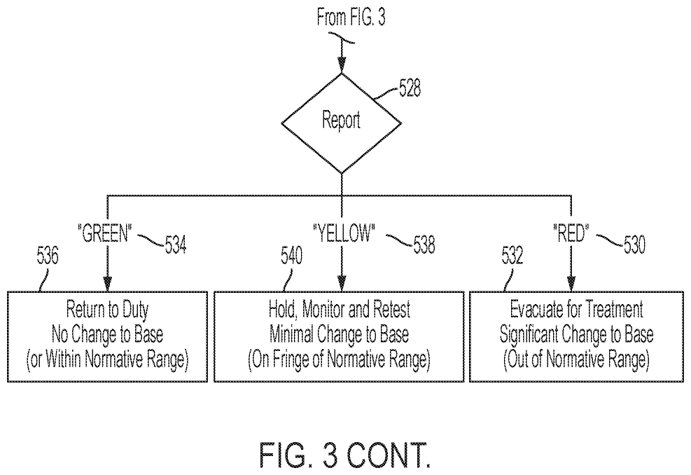

FIG. 3 is a flowchart of operations for employing the OCI system to test a subject and deliver decision-support results for assessing mTBI, according to some embodiments of the present invention. Initially a traumatic event or head injury (i.e., a potential concussive event) occurs to a person (Block 500). The system 10 of FIG. 1 or FIG. 2 is powered on and the electrode cap 400 is attached to the head of the person (Block 502). An electrode impedance check is conducted (Blocks 504-510), and then the OCI test protocol is run (Block 512). This includes olfactory, auditory and somatosensory test protocols. The OCI test protocol involves measuring a continuous, long duration EEG while (1) brief sensory stimuli (odorant pulses, auditory clicks and/or vibratory or electrical pulses to skin) are presented intermittently; and (2) resting EEG (i.e., EEG is recorded without external sensory stimulation) is recorded with eyes shut; (3) resting EEG (i.e., EEG is recorded without external sensory stimulation) is recorded with eyes open.

The odorant delivery device can then be placed under the nose, with the delivery cannulas inserted into the nostrils. A device, such as a Cerescope device, captures and stores all of the cortical waveform data, although other devices may be utilized. The device captures continuous, raw EEG from “go” to “stop”. Any qEEG measure can be “data mined” from this raw waveform. The EEG waveform analytics are then run to verify the quality and usability of the EEG data by assessing the signal-to-noise ratio for different EEG frequency band oscillations by comparing data segments immediately before and immediately following odorant delivery, then computing EEG response signal-to-noise ratio (i.e., OEP or alpha band oscillation signal-to-noise ratio >2 dB) (Block 514). Unusable data are discarded/ignored (Block 516).

Artifact-free, usable data (Block 518) are then submitted to post-processing, in which the features of interest to be used for machine learning-(ML) based classification of mTBI risk (e.g., including, but not limited to cortical EEG, delta band frequency/power, theta band frequency/power, alpha band frequency/power, beta band frequency/power, peak alpha frequency, including ratios between different frequency band power, olfactory OEP and OERP amplitude and latency including different waveform peaks, and alpha band blocking including the latency, frequency and % suppression) are extracted. Data from the initial resting period (two second segments) are projected into the frequency domain using windowed Discrete Fourier transform for each segment, followed by averaging of the resulting single-segment spectra separately for the eye-open and eye-closed portion of the resting period. The resulting segments contain spectral power in electrodes-by-frequencies with frequencies between 1 and 40 Hz in 0.5 Hz steps.

Feature extraction for the resting data is as follows: using principal component analyses (PCA), each subject's power and topography in the canonical delta (˜1 to 4 Hz), theta (4 to 8 Hz), alpha (8 to 13 Hz), and beta ranges (13 to 30 Hz) is extracted, and collapse electrodes over the first spatial principle component for each frequency band. These values are stored for each participant. A priori frequency band selection is cross-validated against label-free segmentation using CARTOOL microstate segmentation. Power in the corresponding CARTOOL-segmented spectral bins is also stored for each subject.

Peak alpha frequency is also estimated using a standard algorithm, and trial-by-trial variability of power at the alpha peak frequency is stored along with the peak frequency. The result is a feature vector of length 10; all ten variables comprising this feature vector have been linked to neurological dysfunction. Embodiments of the present invention will use machine learning to leverage the specific predictive value of these variables regarding mTBI and will complement their information with olfactory-induced changes in brain activity, critical indices of brain dysfunction.

Segments from the olfactory stimulation period undergo two analyses: first, artifact-free trials are averaged and the OEP (latency <200 ms) and oERP (latency >200 ms) measured. Measures are automatized using the same CARTOOL algorithm which identifies subsequent significant deflections and defines their amplitude and latency. Specifically, latency and amplitude of the first two OEP components peaking around 60 ms and 160 ms will be extracted, as well as subsequent P300 and late positive complex responses, a total of eight variables that enter the feature vector for ML. The second analysis is a time-frequency (wavelet-based) decomposition, which focuses on changes in the alpha-band (between 8 and 13 Hz in healthy individuals) power following presentation of the olfactory stimulus. This so-called “alpha-blocking” effect is an index of stimulus perception and represents the abolishment of an idling rhythm in the brain (alpha waves) that is suppressed when an external stimulus is perceived and reflects one of the initial stages of cognitive processing. Alpha blocking responding is diminished by mTBI. Time-frequency decomposition convolves complex Morlet wavelets (gaussian filtered sine and cosine segments tuned to the frequencies of interest) and extracts changes in power relative to the pre-stimulus baseline. Power change information is used to extract the following features for ML: latency of alpha blocking; exact frequency of alpha blocking; and maximum amount of alpha blocking in percent change. Thus, three additional variables enter the feature vector used for ML, for a total of twenty-one variables, spanning a 21-dimensional feature space in which we perform binary kernel-based SVM with ridge regularization (SVM Classifier) (Block 520).

Once the OCI SVM analytics are complete, a subject's current test results can be compared against either a stored demographic normative database (Block 522), and/or compared against the person's own baseline (Block 524). The assessments are then categorized (i.e., as mTBI afflicted or mTBI negative) using SVM (Block 526). EEG features can be extracted for each test subject from the resting EEG and sensory (e.g., olfactory, auditory and/or somatosensory) stimulation periods of the recording session. These features enter the SVM Classifier, which is given labels of healthy vs. mTBI-affected and establishes a hyperplane that separates healthy from mTBI-affected individuals. Specifically, the hyperplane can be trained using a sufficient number of normative EEGs from healthy individuals, with the goal to identify single cases of at-risk or mTBI-afflicted individuals. To that end, ridge regularization can minimize the influence of features with low discriminative value and those with redundant variance. This procedure can be extended to yield predictions regarding multiple clinical categories such as healthy, at risk, or mTBI-afflicted.

A diagnostic report is generated (Block 528), and a determination is made whether the person needs treatment/additional testing or the test results are within an acceptable range. For example, in the illustrated embodiment of FIG. 3, three possible assessments for a military person in an active duty situation are illustrated. If the mTBI diagnosis of the person is within a “normative” range, a “green” flag (Block 534) is generated and the person may be directed to RTD (Return to Duty) (Block 536). If the mTBI diagnosis of the uncertain or somewhat outside of a “normative” range, a “yellow” flag (Block 538) is generated and the person is held for further testing (Block 540). If the mTBI diagnosis of the outside of a “normative” range, a “red” flag (Block 530) is generated and the person is evacuated for treatment (Block 532).

Existing mTBI evaluation methods rely significantly on patient self-reporting, which have limited reliability and external validity; the results of which can be affected by language, education, cognitive and motivational factors. Similarly, measures such as balance testing or eye tracking utilize large, complex instrumentation, require full compliance from the test subject, and lack objectivity as well as reliability. Current objective measures, including blood borne, imaging and qEEG biomarkers lack sufficient sensitivity and specificity to be operationally effective.

Embodiments of the present invention eliminate these shortcomings by establishing an effective and objective OCI of mTBI based on the combination of olfactory (smell) stimulation and resting electrophysiological EEG recordings to detect epileptiform and non-epileptiform abnormal cortical responding, using inexpensive and rapidly-administered, non-invasive electroencephalography (EEG) recordings.

Embodiments of the present invention utilize a novel supported vector machine learning algorithm for diagnosing mTBI, based on a composite index of multiple component neurological biomarkers of mTBI; changes in olfactory system responding and an increased occurrence of cortical epileptiform and non-epileptiform abnormal cortical activity. Olfactory, epileptiform and non-epileptiform abnormal measures have been shown to be sensitive indicators of mTBI, but in combination have yet to be employed clinically. A portable odorant generation and recording system to measure OEPs and oERPs is employed, together with ongoing resting EEG, before, during, and after stimulation. Data from mTBI-naïve subjects will establish a normative data base for comparison analyses with data collected from TBI-diagnosed subjects. This provides the opportunity to confirm the mTBI diagnosis through comparisons of multiple, different objective neurophysiological measures—all mathematically derived from the same, continuous EEG waveform.

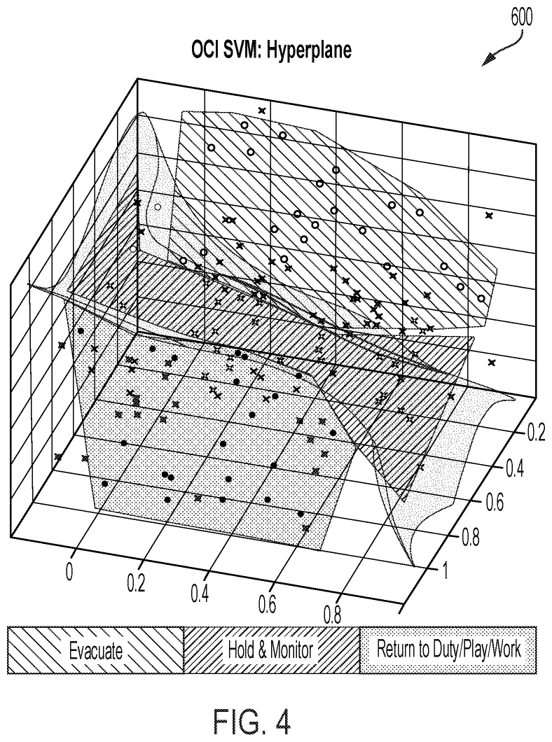

In generating the OCI, EEG features are extracted from the resting and olfactory stimulation periods of a recording session for each participant. These features enter a binary kernel-based support vector machine (SVM), which is given labels of healthy versus TBI at-risk and establishes a hyperplane 600 (FIG. 4) that separates healthy from TBI individuals. The hyperplane is computed in a high-dimensional decision space that is spanned by all the olfactory and EEG indices that are comprised in the Olfaxis Composite Index (OCI). In FIG. 4, a 3-dimensional example is shown only for visualization purposes. Twenty-fold cross-validation is applied throughout development of the hyperplane, to monitor overfitting. The hyperplane 600 is computed using a support vector machine (SVM) algorithm with ridge regularization. The hyperplane 600 represents a decision boundary, where cases on one side are considered affected, and those on the other side unaffected. Ridge regularization decreases the influence of variables that contribute little to correct classification. The axes in the dimensional space represent the normalized (z-transformed) scores that a given individual obtains on each variable of the OCI, e.g. the amplitude of their olfactory evoked potential, the amount of alpha blocking, etc. Cases that lay near the hyperplane are labeled as “hold and monitor”.

Specifically, the hyperplane 600 will be trained using a sufficient number of normative EEGs from healthy individuals with the goal to identify single cases of at-risk or TBI individuals. To that end, ridge regularization will minimize the influence of features with low discriminative value, and those with redundant variance. Leave-one-out validation is used to measure the accuracy of the classifier in identifying single cases as healthy or as TBI. This procedure can be extended to yield predictions regarding multiple clinical categories such as healthy, at-risk, TBI.

Embodiments of the present invention provide the following advances to current devices: 1) the first and only mTBI detection system that employs natural stimulation of a sensory (the olfactory system-measures—one of best indicators of changes in neurologic status) system, then tracks the functional neural activity from the periphery, into the central nervous system; 2) Composite Index of olfactory, cortical epileptiform and non-epileptiform EEG data gives fullest measure of brain function in active and resting phases; 3) Use of supported vector machine learning to determine and utilize most effective biomarkers for detecting/categorizing TBI; 4) portable electrophysiological device for evoked olfactory and resting EEG measures (pre and post-event testing that is unaffected by cognitive abilities, education, language, etc.); 5) international standard 10-20 electrode array allows data collection across the entire cerebral cortex (as opposed to just the frontal cortex) for the most comprehensive, total measure of brain function to facilitate localization of mTBI lesions; 5) 10-20 standard system enables further review by EEG experts, and comparisons with existing databases; 6) results are instantly transmittable to EEG experts for immediate evaluation and consultation by specialists.

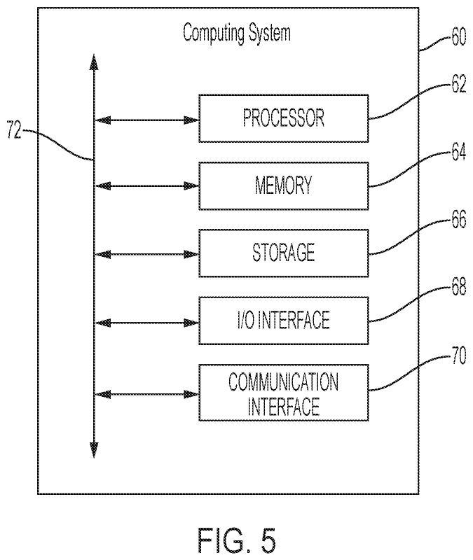

FIG. 5 illustrates an example computing system. In particular embodiments, one or more computer systems 60 perform one or more steps of one or more methods, systems and devices described or illustrated herein. In particular embodiments, one or more computer systems 60 provide functionality described or illustrated herein. In particular embodiments, software running on one or more computer systems 60 performs one or more steps of one or more methods described or illustrated herein or provides functionality described or illustrated herein. Particular embodiments include one or more portions of one or more computer systems 60. Herein, reference to a computer system may encompass a computing device, where appropriate. Moreover, reference to a computer system may encompass one or more computer systems, where appropriate.

This disclosure contemplates any suitable number of computer systems 60. This disclosure contemplates computer system 60 taking any suitable physical form. As example and not by way of limitation, computer system 60 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile computing system, a personal digital assistant (PDA), a server, a tablet computer system, or a combination of two or more of these. Where appropriate, computer system 60 may include one or more computer systems 60; be unitary or distributed; span multiple locations; span multiple machines; span multiple data centers; or reside in a cloud, which may include one or more cloud components in one or more networks. Where appropriate, one or more computer systems 60 may perform without substantial spatial or temporal limitation one or more steps of one or more methods described or illustrated herein. As an example and not by way of limitation, one or more computer systems 60 may perform in real time or in batch mode one or more steps of one or more methods described or illustrated herein. One or more computer systems 60 may perform at different times or at different locations one or more steps of one or more methods described or illustrated herein, where appropriate.

In particular embodiments, computer system 60 includes a processor 62, memory 64, storage 66, an input/output (I/O) interface 68, a communication interface 70, and a bus 72. Although this disclosure describes and illustrates a particular computer system having a particular number of particular components in a particular arrangement, this disclosure contemplates any suitable computer system having any suitable number of any suitable components in any suitable arrangement.

In particular embodiments, processor 62 includes hardware for executing instructions, such as those making up a computer program. As an example and not by way of limitation, to execute instructions, processor 62 may retrieve (or fetch) the instructions from an internal register, an internal cache, memory 64, or storage 66; decode and execute them; and then write one or more results to an internal register, an internal cache, memory 64, or storage 66. In particular embodiments, processor 62 may include one or more internal caches for data, instructions, or addresses. This disclosure contemplates processor 62 including any suitable number of any suitable internal caches, where appropriate. As an example and not by way of limitation, processor 62 may include one or more instruction caches, one or more data caches, and one or more translation lookaside buffers (TLBs). Instructions in the instruction caches may be copies of instructions in memory 64 or storage 66, and the instruction caches may speed up retrieval of those instructions by processor 62. Data in the data caches may be copies of data in memory 64 or storage 66 for instructions executing at processor 62 to operate on; the results of previous instructions executed at processor 62 for access by subsequent instructions executing at processor 62 or for writing to memory 64 or storage 66; or other suitable data. The data caches may speed up read or write operations by processor 62. The TLBs may speed up virtual-address translation for processor 62. In particular embodiments, processor 62 may include one or more internal registers for data, instructions, or addresses. This disclosure contemplates processor 62 including any suitable number of any suitable internal registers, where appropriate. Where appropriate, processor 62 may include one or more arithmetic logic units (ALUs); be a multi-core processor; or include one or more processors 62. Although this disclosure describes and illustrates a particular processor, this disclosure contemplates any suitable processor.

In particular embodiments, memory 64 includes main memory for storing instructions for processor 62 to execute or data for processor 62 to operate on. As an example and not by way of limitation, computer system 60 may load instructions from storage 66 or another source (such as, for example, another computer system 60) to memory 64. Processor 62 may then load the instructions from memory 64 to an internal register or internal cache. To execute the instructions, processor 62 may retrieve the instructions from the internal register or internal cache and decode them. During or after execution of the instructions, processor 62 may write one or more results (which may be intermediate or final results) to the internal register or internal cache. Processor 62 may then write one or more of those results to memory 64. In particular embodiments, processor 62 executes only instructions in one or more internal registers or internal caches or in memory 64 (as opposed to storage 66 or elsewhere) and operates only on data in one or more internal registers or internal caches or in memory 64 (as opposed to storage 66 or elsewhere). One or more memory buses (which may each include an address bus and a data bus) may couple processor 62 to memory 64. Bus 72 may include one or more memory buses, as described below. In particular embodiments, one or more memory management units (MMUs) reside between processor 62 and memory 64 and facilitate accesses to memory 64 requested by processor 62. In particular embodiments, memory 64 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 64 may include one or more memories 64, where appropriate. Although this disclosure describes and illustrates particular memory, this disclosure contemplates any suitable memory.

In particular embodiments, storage 66 includes mass storage for data or instructions. As an example and not by way of limitation, storage 66 may include a hard disk drive (HDD), a floppy disk drive, flash memory, an optical disc, a magneto-optical disc, magnetic tape, or a Universal Serial Bus (USB) drive or a combination of two or more of these. Storage 66 may include removable or non-removable (or fixed) media, where appropriate. Storage 66 may be internal or external to computer system 60, where appropriate. In particular embodiments, storage 66 is non-volatile, solid-state memory. In particular embodiments, storage 66 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 66 taking any suitable physical form. Storage 66 may include one or more storage control units facilitating communication between processor 62 and storage 66, where appropriate. Where appropriate, storage 66 may include one or more storages 66. Although this disclosure describes and illustrates particular storage, this disclosure contemplates any suitable storage.

In particular embodiments, I/O interface 68 includes hardware, software, or both providing one or more interfaces for communication between computer system 60 and one or more I/O devices. Computer system 60 may include one or more of these I/O devices, where appropriate. One or more of these I/O devices may enable communication between a person and computer system 60. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 68 for them. Where appropriate, I/O interface 68 may include one or more device or software drivers enabling processor 62 to drive one or more of these I/O devices. I/O interface 68 may include one or more I/O interfaces 68, where appropriate. Although this disclosure describes and illustrates a particular I/O interface, this disclosure contemplates any suitable I/O interface.

In particular embodiments, communication interface 70 includes hardware, software, or both providing one or more interfaces for communication (such as for example, packet-based communication) between computer system 60 and one or more other computer systems 60 or one or more networks. As an example and not by way of limitation, communication interface 70 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 70 for it. As an example and not by way of limitation, computer system 60 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 60 may communicate with a wireless PAN (WPAN) (such as for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 60 may include any suitable communication interface 70 for any of these networks, where appropriate. Communication interface 70 may include one or more communication interfaces 70, where appropriate. Although this disclosure describes and illustrates a particular communication interface, this disclosure contemplates any suitable communication interface.

In particular embodiments, bus 72 includes hardware, software, or both coupling components of computer system 60 to each other. As an example and not by way of limitation, bus 72 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 72 may include one or more buses 72, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1. A system for measuring neurologic function of a subject, the system comprising:
an odorant generator configured to deliver an odorant stimulation to the subject;
a plurality of electrodes configured to be attached to the subject at respective different locations, wherein the plurality of electrodes are configured to collect neural signals from the subject that are generated by the odorant stimulation; and
a handheld control unit configured to control the odorant generator and to process the neural signals from the plurality of electrodes and generate an assessment of neurologic function of the subject.

2. The system of claim 1, wherein the neural signals comprise neurological measurements of olfactory evoked potentials (OEPs) and olfactory event-related potentials (OERPs).

3. The system of claim 2, wherein the neural signals further comprise neurological measurements of epileptiform and non-epileptiform abnormal cortical activity.

4. The system of claim 1, wherein the odorant generator comprises a handheld intranasal delivery assembly or a mask configured to be placed over a face of the subject.

5. The system of claim 1, wherein the handheld control unit comprises at least one signal processor configured to process the neural signals from the plurality of electrodes and generate the assessment of neurologic function of the subject.

6. The system of claim 1, wherein the odorant generator comprises an odorant cartridge configured to aerosolize a liquid odorant contained therewithin.

7. The system of claim 1, further comprising an auditory generator configured to deliver an audible stimulation to the subject, and wherein the plurality of electrodes are configured to collect neural signals from the subject that are generated by the odorant stimulation and the audible stimulation.

8. The system of claim 7, wherein the handheld control unit is further configured to control the auditory generator to deliver the audible stimulation to the subject.

9. The system of claim 7, wherein the auditory generator is configured to deliver an audible stimulation to the subject via one or more earbuds worn by the subject.

10. The system of claim 1, further comprising a vibrotactile stimulator configured to deliver a somatosensory stimulation to the subject, and wherein the plurality of electrodes are configured to collect neural signals from the subject that are generated by the odorant stimulation and the somatosensory stimulation.

11. The system of claim 10, wherein the handheld control unit is further configured to control the vibrotactile stimulator to deliver the somatosensory stimulation to the subject.

12. The system of claim 10, wherein the vibrotactile stimulator is configured to deliver the somatosensory stimulation to skin of the subject.

13. The system of claim 1, wherein the assessment of neurologic function of the subject comprises comparing a composite index of neural signals for the subject to a previous baseline composite index obtained from the subject.

14. The system of claim 1, wherein the assessment of neurologic function of the subject comprises comparing a composite index of neural signals derived from both sensory-evoked and resting EEG data for the subject to a normative composite index of sensory and cortical measurements of known neurologic function.

15. The system of claim 14, wherein the sensory measurements comprise olfactory, auditory and/or somatosensory measurements.

16. A method of measuring neurologic function of a subject, the method comprising:
delivering an odorant stimulation to the subject via an odorant generator;
collecting neural signals from the subject via a plurality of electrodes attached to the subject, wherein the neural signals are generated by the odorant stimulation; and
processing the neural signals from the plurality of electrodes and generating an assessment of neurologic function of the subject.

17. The method of claim 16, wherein delivering the odorant stimulation and processing the neural signals from the plurality of electrodes is performed by a handheld control unit.

18. The method of claim 16, wherein the neural signals comprise neurological measurements of olfactory evoked potentials (OEPs) and olfactory event-related potentials (OERPs).

19. The method of claim 18, wherein the neural signals further comprise neurological measurements of epileptiform and non-epileptiform abnormal cortical activity.

20. The method of claim 16, wherein delivering the odorant stimulation comprises delivering the odorant stimulation monorhinally or birhinally.

21. The method of claim 16, further comprising delivering an audible stimulation to the subject with the odorant stimulation, and collecting neural signals from the subject generated by the odorant stimulation and the audible stimulation.

22. The method of claim 16, further comprising delivering an somatosensory stimulation to the subject with the odorant stimulation, and collecting neural signals from the subject generated by the odorant stimulation and the somatosensory stimulation.

23. The method of claim 16, wherein processing the neural signals from the plurality of electrodes and generating an assessment of neurologic function of the subject comprises comparing a composite index of neural signals for the subject to a previous baseline composite index obtained from the subject.

24. The method of claim 16, wherein processing the neural signals from the plurality of electrodes and generating an assessment of neurologic function of the subject comprises comparing a composite index of neural signals for the subject to a normative composite index of olfactory and cortical measurements of known neurologic function.

25. A method, comprising utilizing a processor in communication with a tangible storage medium storing instructions that are executed by the processor to perform operations comprising:
accessing sensory evoked response data and EEG data obtained from each of a first plurality of subjects and from each of a second plurality of subjects, wherein the first plurality of subjects are known to have mTBI, and wherein the second plurality of subjects are known to be mTBI unafflicted;
training a machine learning model to identify a brain state associated with mTBI using the sensory evoked response data and EEG data obtained from each of the first plurality of subjects; and
training the machine learning model to identify a brain state associated with non-mTBI using the sensory evoked response data and EEG data obtained from each of the second plurality of subjects.

26. The method of claim 25, wherein the sensory evoked responses are obtained from each of the first and second plurality of subjects via one or more of the following: olfactory stimulation, audible stimulation, somatosensory stimulation.

27. The method of claim 25, wherein training the machine learning model to identify a brain state comprises training the machine learning model to identify epileptiform and non-epileptiform abnormal cortical activity from the EEG data.

28. The method of claim 25, wherein the EEG data comprises resting EEG data.

29. The method of claim 25, further comprising using the machine learning model to identify a brain state of a subject with an unknown mTBI condition.
